(19) United States
(12) Patent Application Publication
(10) Pub. No.: US 2024/0374835 A1
(43) Pub. Date: Nov. 14, 2024

(54) Dose Setting and Drive Mechanism for a Drug Delivery Device

(71) Applicant: Sanofi, PARIS (FR)

(72) Inventors: Zoë Georgina Eardley, Warwick, Warwickshire (GB); Dominic Alexander Graham, Abingdon (GB); Matthew Meredith Jones, Warwick, Warwickshire (GB); William Marsh, Warwick, Warwickshire (GB); Anthony Paul Morris, Warwick, Warwickshire (GB)

(21) Appl. No.: 18/690,462

(22) PCT Filed: Sep. 14, 2022
(86) PCT No.: PCT/EP22/75467

§ 371 Date: Mar. 8, 2024

(30) Foreign Application Priority Data
Sep. 16, 2021 (EP) 21315161.6

(51) Int. Cl. A61M 5/315

(52) U.S. Cl. CPC A61M 5/31553; A61M 5/31526; A61M 5/31585

(57) ABSTRACT

The disclosure relates to a dose setting and drive mechanism for a drug delivery device. In one aspect, the dose setting and drive mechanism comprises a stationary housing, a dial sleeve axially constrained to the stationary housing and rotatable relative to the stationary housing, and a button configured to switch between a dose setting mode and a dose dispensing mode, where the button comprises a first clutch interface configured to rotationally couple and de-couple the button and the stationary housing, and a second clutch interface configured to rotationally couple and de-couple the button and the dial sleeve, a driven member that is axially movable relative to the stationary housing, a drive member axially constrained to the stationary housing and coupled to the driven member in a manner such that rotation of the drive member in a dose dispensing direction is translated into axial displacement of the driven member.

### CROSS REFERENCE TO RELATED APPLICATIONS

The present application is the national stage entry of International Patent Application No. PCT/EP2022/075467, filed on Sep. 14, 2022, and claims priority to Application No. EP 21315161.6, filed on Sep. 16, 2021, the disclosures of which are incorporated herein by reference.

### TECHNICAL FIELD

The present disclosure is generally directed to a dose setting and drive mechanism suitable for use in drug delivery devices.

### BACKGROUND

Pen type drug delivery devices have application where regular injection by persons without formal medical training occurs. This may be increasingly common among patients having diabetes where self-treatment enables such patients to conduct effective management of their disease. In practice, such a drug delivery device allows a user to individually select and dispense a number of user variable doses of a medicament.

There are basically two types of drug delivery devices: resettable devices (e.g., reusable) and non-resettable (e.g., disposable). For example, disposable pen delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism.

These types of pen delivery devices (so named because they often resemble an enlarged fountain pen) generally comprise three primary elements: a cartridge section that includes a cartridge often contained within a housing or holder; a needle assembly connected to one end of the cartridge section; and a dosing section connected to the other end of the cartridge section. A cartridge (often referred to as an ampoule) typically includes a reservoir that is filled with a medication (e.g., insulin), a movable rubber type bung or stopper located at one end of the cartridge reservoir, and a top having a pierceable rubber seal located at the other, often necked-down, end. A crimped annular metal band is typically used to hold the rubber seal in place. While the cartridge housing may be typically made of plastic, cartridge reservoirs have historically been made of glass.

The needle assembly is typically a replaceable double-ended needle assembly. Before an injection, a replaceable double-ended needle assembly is attached to one end of the cartridge assembly, a dose is set, and then the set dose is administered. Such removable needle assemblies may be threaded onto, or pushed (i.e., snapped) onto the pierceable seal end of the cartridge assembly.

The dosing section or dose setting mechanism is typically the portion of the pen device that is used to set (select) a dose. During an injection, a spindle or piston rod contained within the dose setting mechanism presses against the bung or stopper of the cartridge. This force causes the medication contained within the cartridge to be injected through an attached needle assembly. After an injection, as generally recommended by most drug delivery device and/or needle assembly manufacturers and suppliers, the needle assembly is removed and dis-carded.

A further differentiation of drug delivery device types refers to the drive mechanism: There are devices which are manually driven, e.g., by a user applying a force to an injection button, devices which are driven by a spring or the like and devices which combine these two concepts, e.g., spring assisted devices which still require a user to exert an injection force. The spring-type devices involve springs which are preloaded and springs which are loaded by the user during dose selecting. Some stored-energy devices use a combination of spring preload and additional energy provided by the user, for example during dose setting.

EP 2 983 755 B1 discloses a drug delivery device (injection device) comprising a torsion spring which is strained during dose setting and releases stored energy for rotating a piston rod during dose dispensing. The dose setting and drive mechanism of this device comprises an axially displaceable drive sleeve which is in direct engagement with and rotationally constrained to the rotatable piston rod which is provided with a separate bearing (pressure foot). Axial movement of the drive sleeve is caused by axial actuation of a button, thereby switching between a dose setting mode and a dose dispensing mode. A clutch spring is provided in the dose setting and drive mechanism of this known device to apply an axial force to a ratchet feature located between the button and a clutch plate and to bias the clutch plate onto the drive sleeve.

A further spring driven drug delivery device is known from WO 2020/0946699 A1. In this known device, a dial sleeve is provided which is axially movable relative to the housing. Further, a button is provided for switching between a dose setting mode and a dose dispensing mode. This button is axially locked to the dial sleeve but is free to rotate. A piston rod type driven member is rotatable during dose dispensing. A sleeve-shaped clutch element is provided axially and radially spaced from the dial sleeve. This known device does not comprise a clutch spring acting on the clutch element.

### SUMMARY

The present disclosure relates to an improved dose setting and drive mechanism simplifying manufacture and assembly as well as reducing forces for dose dispensing.

According to an aspect of the present disclosure, advantages can be achieved by a dose setting and drive mechanism comprising a stationary housing, a dial sleeve axially constrained to the housing but rotatable relative to the housing, a button operable for switching between a dose setting mode and a dose dispensing mode, a driven member guided axially movable relative to the housing, a drive member axially constrained to the housing and coupled to the driven member such that rotation of the drive member in a dose dispensing direction is translated into axial displacement of the driven member, and a clutch sleeve rotationally constrained but axially movable relative to the drive member. A first clutch interface may be provided between the button and the housing, a second clutch interface may be provided between the button and the dial sleeve, a third clutch interface may be provided between the drive member or the clutch sleeve and the housing and a fourth clutch interface may be provided for rotationally coupling and de-coupling the clutch sleeve and the dial sleeve. According to this aspect of the disclosure, in the dose setting mode, the button is rotationally de-coupled from the housing by the first clutch interface, the button is rotationally coupled to the dial sleeve by the second clutch interface, and the clutch sleeve is de-coupled from the dial sleeve by the fourth clutch interface. In addition, in the dose dispensing mode, the button is rotationally coupled to the housing by the first clutch interface, the button is rotationally de-coupled from the dial sleeve by the second clutch interface, and the clutch sleeve is coupled to the dial sleeve by the fourth clutch interface.

The stationary housing may be an outer housing shell or body partially receiving and/or encasing e.g., the dial sleeve, the drive member, the driven member and the clutch sleeve. The housing may have at least one, e.g., elongate, window or opening permitting to see at least a portion of an internal component part of the dose setting and drive mechanism, e.g., a portion of the dial sleeve.

The, e.g., outer, housing of the mechanism may be a housing of a drug delivery device and may define a central axis extending between a distal end and a proximal end. “Distal” or “lower” is used herein to specify directions, ends or surfaces which are arranged or are to be arranged to face or point towards a dispensing end of the dose setting and drive mechanism when used in an injection device or of the drug delivery device or components thereof and/or point away from, are to be arranged to face away from or face away from the proximal end. On the other hand, “proximal” or “upper” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example. The terms “axial”, “radial”, or “circumferential” as used herein may be used with respect to a main longitudinal axis of the device, the cartridge, the housing or the cartridge holder, e.g., the axis which extends through the proximal and distal ends of the cartridge, the cartridge holder or the drug delivery device.

The dial sleeve may be a number sleeve with a series of numbers or symbols on an outer surface which may be used to indicate the amount of a selected dose, e.g., when visible through a window or opening in the housing. The dial sleeve may be provided with an opening for attaching a hook shaped portion of a torsion spring. The dial sleeve may comprise at least two separate component parts, e.g., a lower or distal portion and an upper or proximal portion, which may be rigidly constrained to each other during assembly such that they move together like a unitary component part. For example, the distal portion of the dial sleeve may be a substantially cylindrical sleeve comprising at least a portion, e.g., its proximal portion, having an outer helical thread. A circular bead or groove may be provided for snap engagement in order to axially constrain the distal portion of the dial sleeve. The proximal portion of the dial sleeve may be ring shaped. The proximal portion may comprise at least one ring of teeth for rotationally coupling the dial sleeve to a further component, e.g., forming a clutch interface for rotationally coupling and de-coupling the button and the dial sleeve. The distal portion and the proximal portion may be provided with corresponding engagement features for rigidly connecting the distal portion and the proximal portion to form the dial sleeve. An inner portion of the dial sleeve may be provided with a series of teeth or with one or more splines and/or grooves for engaging corresponding features of the clutch sleeve.

The button may be located at the proximal end of the dose setting and drive mechanism and/or may form of the proximal end of a drug delivery device comprising such a dose setting and drive mechanism. The button may have a proximally facing surface for actuation by a user and an, e.g., central, stem extending distally from this surface. A series of teeth may extend distally from the proximal surface, e.g., a partial or full ring of teeth, forming a first clutch interface for rotationally coupling and de-coupling the button and the housing. The additional series of teeth may be arranged radially outside of the stem. The stem may be provided with a further series of teeth for rotationally coupling the button to a further component, e.g., forming a second clutch interface.

The driven member may be a threaded lead screw having at least one axially extending guiding feature, e.g., a slot and/or a rib. When assembled, the driven member may be guided in a corresponding guiding feature of the housing, for example an insert of the housing, such that the driven member is axially displaceable with respect to the housing but rotationally constrained to the housing. The threaded portion of the driven member engages a corresponding thread, e.g., an inner thread, of the drive member. The distal end of the driven member may have an enlarged diameter forming a pressure foot for abutting a rubber bung in a cartridge. In other words, the pressure foot is an integrally formed portion of the driven member rigidly connected thereto.

The dose setting and drive mechanism may further comprise a clutch spring interposed between the drive member and the clutch sleeve such that the clutch sleeve is biased proximally with respect to the drive member. For example, the clutch spring may be situated inside the clutch sleeve and may act axially between the drive member and the clutch sleeve. Frictional losses between the drive member and the housing may be reduced since the clutch spring diverts some of the axial reaction force applied by the driven member to the drive member during dispense through the interface between the clutch sleeve and the button which has low frictional loss as a contact spigot may have a small diameter. In other words, in an example of the dose setting and drive mechanism according to the present disclosure, an axial force acting on the driven member from the bung in the cartridge during dose dispensing is at least partially transferred from the driven member via the threaded interface to the drive member, is transferred from the drive member via the clutch spring to the clutch sleeve and is reacted via a spigot contact interface to the button, wherein the diameter of the spigot contact interface between the button and the clutch sleeve is smaller than the outer diameter of the drive member.

The drive member may be a tubular element, e.g., in the form of an elongate sleeve or in the form of a nut. The drive member is provided with an internal thread for engaging the threaded driven member. For example, the driven member may be rotationally constrained to the housing and coupled to the drive member by a threaded interface. The drive member may be provided with a radially protruding outer flange for axially constraining the drive member to the housing. Further, the drive member may comprise one or more outer splines and/or grooves for rotationally coupling the drive member to the clutch sleeve. In more detail, the drive member may be permanently rotationally constrained to the clutch sleeve but may be axially displaceable with respect to the clutch sleeve. A compression spring, e.g., the clutch spring, may abut a proximal end of the drive member. In addition, the drive member or the clutch sleeve may comprise flexible arms or a series of outer splines or teeth forming a third clutch interface for preventing rotation of the drive member and the clutch sleeve relative to the housing in a direction opposite the dose dispensing direction. Still further, a dispense clicker may be formed by a flexible arm on the drive member which interacts with the body during dispense. This interface may also be reverse loaded during the last dose stop loading.

The clutch sleeve is a tubular element provided with one or more internal splines and/or grooves, e.g., at its distally facing and portion, for engaging corresponding splines and/or grooves of the drive member. In other words, the drive member and the clutch sleeve may comprise axially extending grooves and splines for permanently rotationally constraining the clutch sleeve to the drive member while permitting relative axial movement therebetween. The clutch sleeve may have an internal seat, e.g., a recess, for receiving and abutting a compression spring, e.g., the clutch spring. In addition, the clutch sleeve may be provided with one or more external teeth or a series of splines and/or grooves forming a fourth clutch interface for releasably rotationally engaging the dial sleeve. In other words, the clutch sleeve may be rotationally coupled and decoupled with respect to the dial sleeve by an axial displacement of the clutch sleeve with respect to the dial sleeve.

In the dose setting and drive mechanism, distal movement of the button is transmitted to the clutch sleeve and proximal movement of the clutch sleeve is transmitted to the button. This allows movement of the clutch sleeve for coupling and de-coupling the dial sleeve.

The dose setting and drive mechanism may further comprise a drive spring, e.g., a torsion spring interposed between the housing and the dial sleeve. In this respect, the drive spring may be directly attached to the dial sleeve. Further, the drive spring may be attached to the housing of via an insert which is at least rotationally permanently constrained to the housing. As an alternative, the drive spring may be directly attached to the housing. In other words, rotation of the dial sleeve with respect to the housing in a first direction, e.g., increasing the set dose, strains the torsion spring, whereas rotation of the dial sleeve with respect to the housing in a second direction opposite to the first direction, e.g., decreasing the set dose, releases the torsion spring. The torsion spring may be prestressed such that a torque is applied to the dial sleeve by the torsion spring in a zero unit (OU) position of the dial sleeve with respect to the housing, e.g., when no dose has been set.

The dose setting and drive mechanism may comprise a separate dial grip operable by a user to set a dose. The button may be rotationally constrained but axially displaceable relative to the dial grip. Further, the dial grip may be axially constrained to the housing. In some embodiments, a fifth clutch interface comprising an overhaulable ratchet element is provided for rotationally coupling the housing and a dial grip, wherein, in the dose setting mode, the driving torque or driving force of the drive spring is reacted via the fifth clutch interface. In other words, this fifth clutch interface prevents unintended dose dispensing movements of the component parts when a dose has been set. Nevertheless, the fifth clutch interface may be overhauled in both directions (increasing or decreasing a set dose) by a user applying a dose setting torque to the dial grip.

According to a further independent aspect of the present disclosure, the dose setting and drive mechanism may further comprise a one-way ratchet, e.g., in the form of a housing (or body) insert rotationally constrained to the housing, wherein the one-way ratchet is the third interface allowing rotation of the drive member relative to the housing in the dose dispensing direction but preventing rotation of the drive member relative to the housing in the direction opposite the dose dispensing direction. Further, the fourth clutch interface for rotationally coupling and de-coupling the clutch sleeve and the dial sleeve may comprise a ring of teeth formed on the clutch sleeve and a corresponding ring of teeth formed on the dial sleeve.

Still further, in addition or as an alternative to the above mentioned one-way ratchet, a dialing detent may be provided. In more detail, the ratchet element may be a metal pressing, for example a bent strip rotationally constrained to the housing with one or more detents for engaging corresponding detent positions defined in the dial grip.

In this example, the button may directly abut the clutch sleeve. Thus, a separate clutch plate between the button and the clutch sleeve as present in prior art devices may be omitted.

According to a further independent aspect of the present disclosure, the dose setting and drive mechanism may further comprise a toothed ring rotationally constrained to the housing, wherein the toothed ring is the third interface rotationally coupling the drive member to the housing in the dose setting mode and rotationally de-coupling the drive member from the housing in the dose dispensing mode. The toothed ring may be an insert at least rotationally fixed in the housing. Further, the fourth clutch interface for rotationally coupling and de-coupling the clutch sleeve and the dial sleeve may comprise a separate ring which is rotationally constrained to the dial sleeve and comprises a ring of teeth for engaging a corresponding ring of teeth formed on the clutch sleeve. The separate ring may be configured as a clutch plate arranged on the proximal end of the clutch sleeve. In this example, the button may indirectly abut the clutch sleeve via the separate ring (clutch plate).

According to a further aspect of the present disclosure a drug delivery device, e.g., a disposable drug delivery device, comprises the above defined dose setting and drive mechanism. The drug delivery device may further comprise a container receptacle which is permanently attached to the dose setting and drive mechanism. As an alternative, the container receptacle may be releasably attached to the dose setting and drive mechanism. The container receptacle is adapted to receive a container, e.g., a cartridge, containing a medicament.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-pal-mitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eli-gen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Chorion-gonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the abovementioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves, for a example do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF FIGURES

Non-limiting, exemplary embodiments of the invention will now be described with reference to the accompanying drawings, in which:

FIG. 1 shows a perspective view of a drug delivery device according to a first embodiment of the present disclosure.

FIG. 2 shows an exploded view of the drug delivery device of FIG. 1.

FIG. 3 shows a sectional view of the drug delivery device of FIG. 1.

FIG. 4 shows a sub-assembly the drug delivery device of FIG. 1.

FIGS. 5a, b show sectional views of the dose setting and drive mechanism of the drug delivery device of FIG. 1 in an at rest condition (FIG. 5a) and in a button depressed condition (FIG. 5b).

FIG. 6 show the spline insert and the housing of the device of FIG. 1.

FIG. 7 shows in cross section the housing, the ratchet element and the dial grip of the device of FIG. 1.

FIG. 8 shows the body insert, the drive member and the clutch sleeve of the device of FIG. 1.

FIG. 9 shows the body insert of the device of FIG. 1.

FIGS. 10a, b show in partial cross section a portion of the drug delivery device of FIG. 1 in an at rest condition (FIG. 10a) and in a button depressed condition (FIG. 10b).

FIG. 11 shows an exploded view of a drug delivery device according to a second embodiment of the present disclosure.

FIG. 12 shows a sectional view of the drug delivery device of FIG. 11.

FIG. 13 shows the clutch plate of the drug delivery device of FIG. 11.

FIGS. 14a, b show in cross section a portion of the drug delivery device of FIG. 11 in an at rest condition (FIG. 14a) and in a button depressed condition (FIG. 14b).

### DETAILED DESCRIPTION

In the figures, identical elements, identically acting elements or elements of the same kind may be provided with the same reference numerals.

In the following, some embodiments will be described with reference to an insulin injection device. The present disclosure is however not limited to such application and may equally well be deployed with injection devices that are configured to eject other medicaments or drug delivery devices in general, including pen-type devices and/or injection devices.

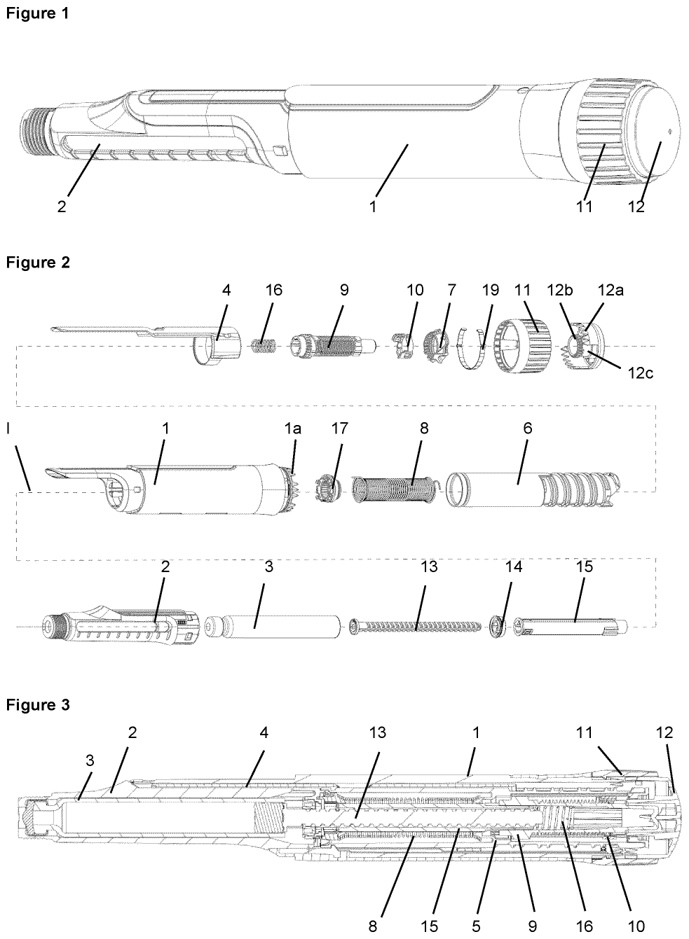

FIGS. 1 to 3 show a perspective view, an exploded view and a cross-sectional view of the medicament delivery device or drug delivery device comprising a dose setting and drive mechanism. As can be taken from FIG. 3, component parts of the drug delivery device and of its dose setting and drive mechanism are located concentrically about a common principal longitudinal axis I. in the following, axial movement is to be understood as a movement parallel to axis I, a rotation is to be understood as a rotation about the axis I and a lateral movement is to be understood as a movement perpendicular to axis I.

The depicted drug delivery device comprises a stationary housing 1 (body), a cartridge holder 2 for retaining a cartridge 3, a gauge element 4, a dial sleeve 5 in the form of a number sleeve and comprising a lower (distal) portion 6 and an upper (proximal) portion 7, a drive spring 8 in the form of the torsion spring, a clutch sleeve 9, a nut 10, a dial grip 11, a button 12, a driven member 13 in the form of a lead screw, a spline insert 14, a drive member 15, a clutch spring 16, a body insert 17 comprising a one-way ratchet 18 and a ratchet element 19 in the form of a detent pressing. In addition, the drug delivery device may further comprise a removable cap (not shown) covering the cartridge holder 2.

The button 12 is permanently splined to the dial grip 11. It is also splined to the upper portion 7 of the dial sleeve 5 when the button is not pressed but this splined interface is disconnected when the button 12 is pressed (second clutch interface). When the button 12 is pressed, ramp-like splines 12a on the button 12 engaged with corresponding ramp-like splines 1a on the housing 1 (FIG. 2), preventing rotation of the button 12 and hence the dial grip 11 during dose dispensing (first clutch interface). These splines disengage when the button 12 is released allowing a dose to be dialed. An additional set of teeth 12b is provided on an inner stem 12c of the button 12 which—depending on the axial position of the button 12 relative to the housing 1—engage or disengage with a corresponding ring of teeth on the dial sleeve 5, more specifically on the upper portion 7 of the dial sleeve 5 (second clutch interface). A central spigot 12d extends distally from a proximal end face of the button 12 (FIG. 5b). The central spigot is adapted to contact a spigot contact face 9b formed in the proximal end face of the clutch sleeve 9. In the exemplary embodiment of FIG. 8, the spigot contact face 9b is formed as a central depression in the proximal end face of the clutch sleeve 9. Due to the small diameter of the spigot 12d and the spigot contact face 9b, relative rotation in the interface between the spigot 12d and the spigot contact face 9b causes relatively low friction torque.

The dial grip 11 is axially constrained to the housing 1 but is allowed to rotate with respect to the housing 1. However, ratchet element 19 interposed between the dial grip 11 and the housing prevents free rotation between the dial grip and the housing. Rather, a certain amount of torque has to be applied to overhaul this ratchet element 19. The dial grip 11 has a toothed inner surface 11a engaging the ratchet element 19 (FIG. 7). The dial grip 11 is rotationally constrained via the splined interface to the button 12.

The ratchet element 19 is rotationally constrained to the housing 1 by to hook features 19a (FIG. 7) at each end and axially constrained between the housing 1 and the dial grip 11. Two nib features 19b engage with ramped surfaces on the inside of the dial grip to form a dialing clutch. Two large pockets 1b in the housing 1 behind each nib 19b allow radial movement of the nib during overhaul. This dialing detent or ratchet (fifth clutch interface) allows the user to dial up and down doses whilst retaining the mechanism against the torque of the drive spring 8.

The lower portion 6 of the dial sleeve 5 is rigidly fixed to the upper portion 7 of the dial sleeve 5 during assembly to form a number sleeve. These components are separate components that simplify mould tooling and assembly. This sub-assembly is constrained to the housing 1 by features towards the distal end to allow rotation but not axial translation. The lower portion 6 is marked with a sequence of numbers which are visible through the gauge component 4 and a window in the housing 1 to denote the dialed dose of a medicament.

The gauge component 4 is constrained to prevent rotation but allow axial translation relative to the housing 1 via a splined interface. The gauge component 4 has a helical feature on its inner surface which engages with a helical thread formed in the lower portion 6 of the dial sleeve 5 such that rotation of the dial sleeve 5 causes axial translation of the gauge component 4. This helical feature on the gauge component also creates abutments against the end of the helical thread in the dial sleeve 5 to limit the minimum and maximum dose that can be set.

The nut 10 provides an optional last dose stop feature and is located between the dial sleeve 5 and the clutch sleeve 9. It is rotationally constrained to the dial sleeve 5 via a splined interface. It moves along a helical path on and relative to the clutch sleeve 9 via a threaded interface when relative rotation of occurs between the dial sleeve 5 and the clutch sleeve 9 which occurs during dialing only.

The clutch sleeve 9 is axially biased against the button 12 by proximal action of the clutch spring 16. A splined tooth interface with the dial sleeve 5 is not engaged during dialing but engages when the button 12 is pressed, preventing relative rotation between the clutch sleeve 9 and the dial sleeve 5 during dispense (fourth clutch interface). A further splined interface is permanently engaged with the drive member 15, allowing relative axial travel between the clutch sleeve 9 and the drive member 15 whilst maintaining rotational coupling.

The drive member 15 is rotationally splined to the clutch sleeve 9 (FIG. 8) and axially constrained between the housing 1 and the spline insert 14. The clutch spring 16 acts distally on the drive member 15. Flexible arms protrude from the drive member 15 into ratchet teeth on the body insert 17 forming the one-way ratchet 18 to produce an audible feedback during dispense whilst providing a stop abutment when the last dose stop is engaged.

The body insert 17 is rigidly fixed to the housing 1 and provides an anchor for one end of the drive spring 8. It also contains ratchet teeth to interact with the flexible arms of the drive member 15 (third clutch interface).

The drive spring 8 is attached at one and to the body insert 17 and at the other end to the dial sleeve 5. The drive spring is pre-wound upon assembly such that it applies a torque to the dial sleeve 5 when the mechanism is at zero units dialed. The action of rotating the dial grip 11 to set a dose rotates the dial sleeve 5 relative to the housing 1 and charges the drive spring 8 further.

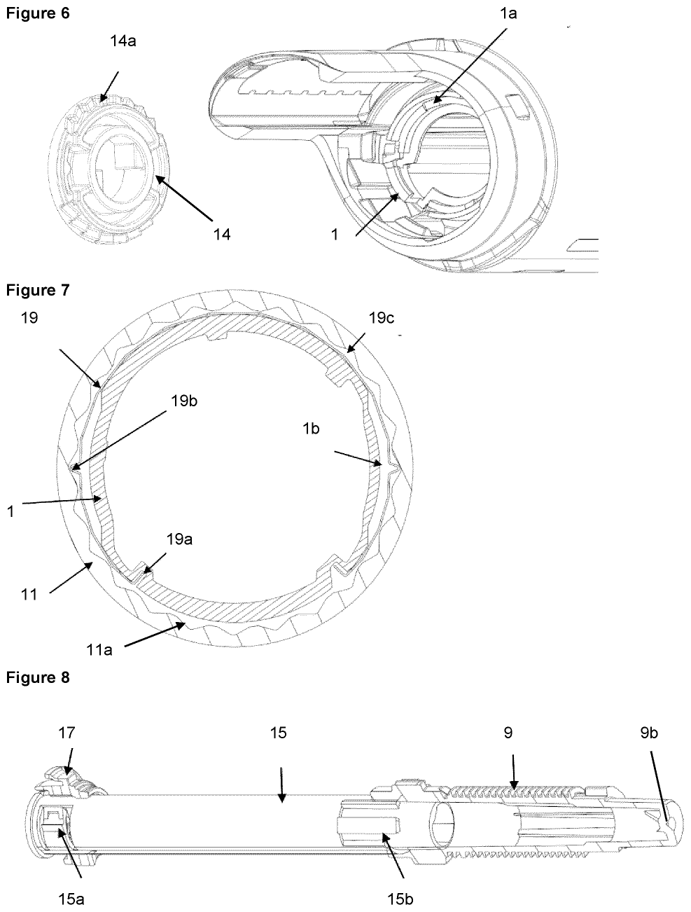

The spline insert 14 is rigidly fixed to the housing 1 and has a splined interface rotationally constraining the driven member 13. For example, as depicted in FIG. 6, it may be assembled in multiple different rotational positions. This reduces the effect of component and assembly tolerances on the priming and therefore reduces the number of prime doses.

The driven member 13 is rotationally constrained to the spline insert 14 via a splined interface and is threaded to the drive member 15. When the drive member 15 is rotated, the threaded engagement is overhauled causing the driven member 13 to be moved axially relative to the spline insert 14. Since the driven member 13 does not rotate relative to the liquid medicament cartridge 3, the bung contact surface (pressure foot) may be integrated into the base of the member 13.

The axial position of the clutch sleeve 9 and the button 12 is defined by the action of the clutch spring 16 which applies a force on the clutch sleeve 9 in the proximal direction. This spring force is reacted via the clutch sleeve 9 and the button 12 and in the at rest position of the mechanism it is the reacted through the dial grip 11 to the housing 1. The spring force ensures that the spigot formed at the proximal end of the clutch sleeve 9 is engaged with the button 12. In the at rest position it also ensures that the splines of the button 12 are engaged with the dial sleeve 5.

The housing provides location for the liquid medicament cartridge 3 and the cartridge holder 2, windows for viewing the dose number and the gauge component 4 and a feature on its external surface to retain the dial grip 11. In the depicted embodiments, the drug delivery device is a disposable device, e.g., the cartridge holder is permanently fixed to the housing 1 not permitting relacing an empty cartridge 3 by new one.

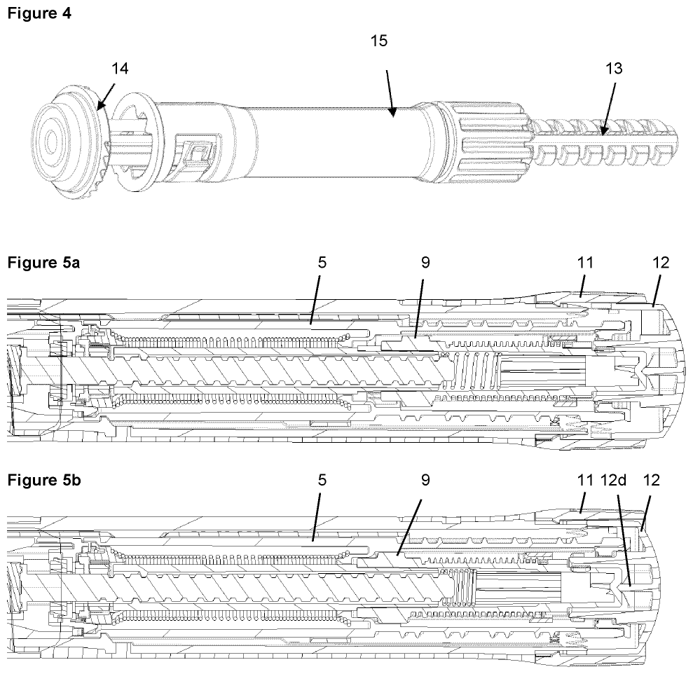

The driven member 13, the spline insert 14 and the drive member 15 are depicted in FIG. 4 as a sub-assembly which may be assembled into the housing 1. In this sub-assembly, the driven member 13 is pre-threaded to the drive member 15 with the spline insert 14 rotationally engaged to the driven member 13. This arrangement of components allows for an improvement in the priming gap of the device. A priming gap is introduced between the bung contact pad (pressure foot) of the driven member 13 and the bung in the cartridge 3 to ensure that the bung cannot be compressed by the driven member 13 in tolerance situations. For example, at first usage of the device, the driven member 13 is advanced to close up this priming gap for medicament is dispensed from the cartridge 3. A smaller priming gap is desirable for improved usability and device confidence.

The relative position (rotational and axial) of the driven member 13 and drive member 15 is carefully controlled during assembly. As they are advanced into the housing 1 the spline insert 14 is clipped proximally into the housing 1 where it is axially retained by clips. Rotationally, it engages with its set of teeth 14a with one of a set of ramped teeth 1a formed in the housing 1 whichever is closest to the orientation in which it is presented. This method of the assembly allows for a reduced priming gap since the axial location of the contact pad is directly controlled by assembly equipment, rather than through long rotational tolerance chains, and the variation in the rotational position of the spline insert is limited to the pitch between the ramped teeth.

The user selects a variable dose of liquid medicament by rotating the dial grip 11 clockwise. The first clutch interface 12a between the button 12 and the housing 1 is de-coupled permitting relative rotation between these components. The dial grip 11 is splined to the button 12 which is in turn splined to the dial sleeve 5 via teeth 12b during dose selection (second clutch interface coupled). Therefore, rotation of the dial grip 11 generates an identical rotation of the dial sleeve 5. As the splines 9a of the clutch sleeve 9 are axially spaced and disengaged from the corresponding splines of the dial sleeve 5 (third clutch interface de-coupled) rotation of the dial sleeve 5 is not transmitted to the clutch sleeve 9 and the drive member 15. In addition, the drive member 15 is prevented from rotation in a direction opposite to the dispensing direction by one-way ratchet 18 (fourth clutch interface).

Rotation of the dial sleeve 5 causes charging of the drive spring 8 increasing the energy stored within it. As the dial sleeve 5 rotates, the gauge component 4 translates axially due to its threaded engagement thereby showing the value of the dialed dose. The gauge component 4 has flanges either side of the window area which cover the numbers printed on the dial sleeve 5 adjacent to the dialed dose to ensure the set dose number is made visible to the user. The mechanism utilizes a dial grip with an increased diameter relative to the housing 1 which aids dialing although it is not a requirement of the mechanism. This feature is particularly useful but not essential for an auto-injector mechanism where a power supply is charged during dose setting and the torque to turn the dial grip may be higher than for non-auto injector devices.

The clutch sleeve 9 is prevented from rotating as the dose is set and the dial sleeve 5 rotated due to the splined engagement to the drive member 15 which in turn is prevented from rotating by the dispense clicker engagement (one-way ratchet 18 of third clutch interface) with the body insert 17 and frictional interface with the spline insert 14 under of the action of the axial force from the clutch spring 16.

Relative rotation of occurs between the dial grip 11 and the ratchet element 19 (detent pressing) via the ratchet interface. The user torque to rotate the dial grip is the sum of the torque to wind up the drive spring 8 and the torque to overhaul the dialing clutch (fifth clutch interface) on the dial grip 11. The torque to overhaul the dialing clutch is a function of the radial loads applied by the ratchet element 19, the clockwise ramp angle of the clutch teeth on the dial grip 11, the friction coefficient between the mating surfaces, the mean radius of the clutch features and size and orientation of the pockets 1b and surfaces in the housing 1 (FIG. 7). The ratchet element 19 (detent pressing) is designed to provide a tangential opposing force through its nibs to the dial grip detent features, acting to oppose overhaul of the ratchet feature. The angle of the nib 19b is much steeper than the dial grip ramps to ensure a cam follower arrangement between the ratchet element 19 and the dial grip 11 since this minimizes the effect of tolerance deviations in manufacture of the ratchet element 19. This also allows torque to be modified without changing the ratchet element 19 since there is scope to increase or decrease the angle of the ramps of the dial grip 11. The facetted design with bends 19c of the pressing forming the ratchet element 19 provides a more consistent bend resulting in less component variation.

In order to provide a more consistent dialing feedback to the user in both dialing directions (both with and against action of the drive spring 8), it is beneficial for the dialing clutch to have a different overhaul torque in each dialing direction to offset the action of the drive spring 8. This can be implemented by modification of the size and orientation of the pockets 1b and hook surfaces in the housing 1.

The ratchet element 19 biases into the housing 1 in the direction of torque applied by dial grip 11, causing contact at the leading edge of the pocket 1b in the housing 1. The location of the edge of the pocket in housing 1 determines the effective length of the ratchet element 19 and therefore its stiffness during overhaul. Moving the edges of the pockets closer to the nibs 19b has the effect of increasing the stiffness of the ratchet element 19 and therefore increasing overhaul torque. Since the pocket edges can be independently controlled for both dialing directions, it is possible to incorporate pockets 1b asymmetrically located about the nibs 19b in order to produce different overhaul torques in each dialing direction.

A further embodiment for producing a different overhaul torque in each dialing direction is to provide different end constraints to the ratchet element 19. This may be implemented by locating one of the hooks in a blind pocket, such that both tensile and compressive loadings can be reacted by the housing 1, whilst locating the adjacent hook in a tensile-only slot. In the dial up direction, the ratchet element 19 is biased clockwise, causing a single hook to contact the housing 1 where it is reacted with a tensile load. A tensile load on the ratchet element 19 tends to draw spring material radially inwards, reducing reaction force and nib engagement and therefore decreasing overhaul torque. In the dial down direction, the ratchet element 19 is biased anti-clockwise, resulting in both hooks contacting the housing 1. Having two contact points helps to constrain the ratchet element 19, increasing its reaction force to the dial grip 11 and therefore increasing overhaul torque. Additionally, providing a compressive reaction to the ratchet element 19 tends to bias spring material outwards into the dial grip 11, increasing the reaction force and engagement of the nib and therefore increasing overhaul torque.

As the user rotates the dial grip 11 sufficiently to increment the mechanism by 1 increment the dial sleeve 5 rotates relative to the clutch sleeve 9 by one ratchet tooth. At this point the nibs 19b of the ratchet element 19 re-engage into the next detented position 11a. An audible click is generated by the ratchet re-engagement, and tactile feedback is given by the change in torque input.

An alternative embodiment of the ratchet element 19 has a single nib and has less angular wrap of the housing 1. This may be less sensitive to tolerance variation between the ratchet element 19 and housing 1 and is also simpler to manufacture than a ratchet element 19 with an almost full wrap. This still allows for asymmetrically orientated pockets in the housing 1 and different hook contacts to provide independently controllable dialing torques for each direction.

Relative rotation of the dial sleeve 5 and the clutch sleeve 9 also causes the last dose nut 10 to travel along its threaded path, towards its last dose abutment on the clutch sleeve 9.

With no user torque applied to the dial grip 11, the dial sleeve 5 is now prevented from rotating back under the torque applied by the drive spring 8, solely by the ratchet engagement between the dial grip 11 and the housing 1 via the detent pressing of ratchet element 19 (fifth clutch interface). Additional energy is stored within the drive spring 8 for each dose increment and audible and tactile feedback is provided for each increment dialed by the re-engagement of the ratchet teeth. The torque to rotate the dial grip 11 increases as the torque to wind up the torsion drive spring increases. The torque to overhaul the ratchet in the anti-clockwise direction can (independently of the torque provided in this direction by the drive spring 8) therefore be greater than the torque applied to the dial sleeve 5 by the drive spring 8 when the maximum dose has been reached.

If the user continues to increase the selected dose until the maximum dose limit is reached, the dial sleeve 5 engages with its maximum dose abutment on the gauge component 4. This prevents further rotation of the dial sleeve 5 and dial grip 11.

Depending on how many increments have already been delivered by the mechanism, during selection of a dose, the last dose nut 10 may contact its last dose abutment with the clutch sleeve 9. This abutment prevents further relative rotation between the dial sleeve 5 and the clutch sleeve 9, thereby preventing a rotation increasing the set dose.

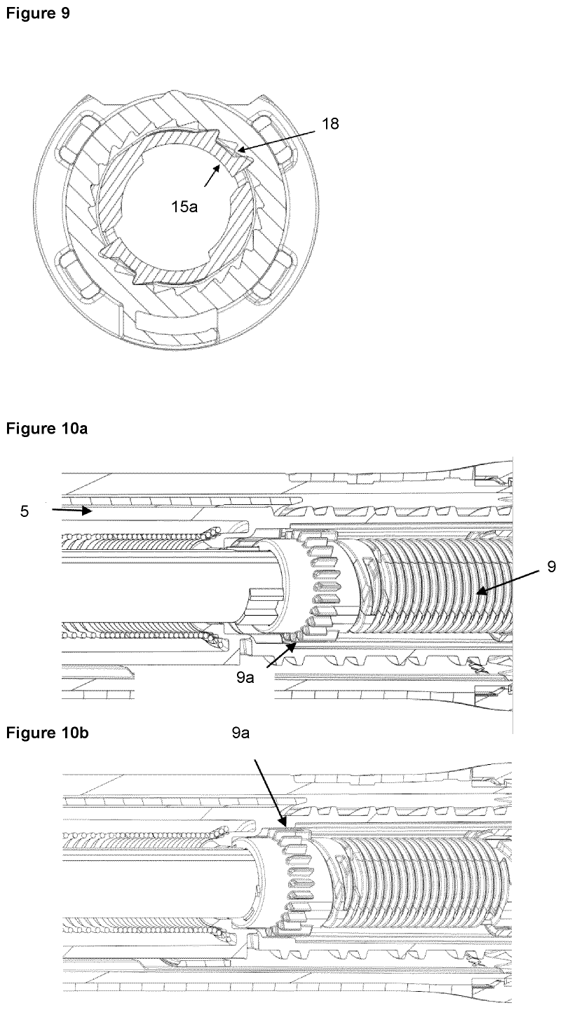

The clutch sleeve 9 is splined to the drive member 15 by splines 15b (FIG. 8) to prevent relative rotation. Flexible arms 15a located at the distal end of the drive member 15 engage with a ramped tooth ratchet interface (one-way ratchet 18) on the body insert 17, itself rigidly fixed to the housing 1. Thus, this third clutch interface prevents rotation of the drive member 15 in a direction opposite to the dose dispensing direction. In the last dose stop engagement condition the interface between the drive member 15 and body insert 17 is loaded in the dose selection direction. In this loading configuration the flexible arms 15a on the drive member 15 abut against steep surfaces on the body insert 17 and therefore relative rotation is prevented between these two components. The angular width of ramped teeth 17a of the body insert 17 is selected to match that of a single dose increment, ensuring that the drive member 15 is in a position to prevent dialing at the last dose abutment. In the depicted embodiment, the flexible arms 15a may be tangentially orientated with two discrete radially protruding tips which contact the ramped tooth interface (FIG. 9), one tip is at the end of the arm (“end tip”), whilst the other is approximately halfway along its length (“mid tip”) and does not protrude as far radially outwards. During loading in the last dose stop condition, the end tip first abuts against the ramped interface before a small amount of local deflection causes the mid tip to also abut. Since the mid tip is closer to the root of the arm, stiffness and failure torque is increased over using a single end tip.

With the mechanism in a state in which a dose has been selected, the user is able to deselect any number of increments from this dose. Deselecting a dose is achieved by the user rotating the dial grip 11 anti-clockwise. The torque applied to the dial grip 11 by the user is sufficient, when combined with the torque applied by the drive spring 8, to overhaul the ratchet between the ratchet element 19 and the dial grip 11 in the anti-clockwise direction. When the ratchet is overhauled, anti-clockwise rotation occurs in the dial sleeve 5, which returns the dial sleeve 5 towards the zero dose position, and unwinds the drive spring 8. The relative rotation between the dial sleeve 5 and clutch sleeve 9 causes the last dose nut 10 to return along its helical path, away from the last dose abutment.

With the mechanism in a state in which a dose has been selected, the user is able to activate the mechanism to commence delivery of a dose. Delivery of a dose is initiated by the user pressing the button 12 axially (FIGS. 5a, 5b, 10a, 10b). When the button 12 is depressed, splines 12a on the button engage with splines on the housing 1 preventing rotation of the button 12 and hence the dial grip 11 during dispense e.g., the first clutch interface is coupled. Further, splines 9a of the clutch sleeve 9 engage with corresponding splines of the dial sleeve 5 such that torque may be transferred from the dial sleeve 5 to the clutch sleeve 9 and hence to the drive member 15, e.g., the fourth clutch interface is closed. Rotation of the drive member 15 in the dose dispensing direction is permitted by the third clutch interface, e.g., one-way ratchet 18 of the body insert 17.

Finally, teeth 12b between the button 12 and the dial sleeve 5 are disengaged, e.g., the second clutch interface is de-coupled, rotationally disconnecting the button 12 and the dial grip 11 from the components involved in dose dispensing, e.g., the drive mechanism components including the dial sleeve 5, the clutch sleeve 9, the drive member 15 and the driven member 13. Thus, the clutch sleeve 9 can now rotate and is driven by the drive spring 8 via the dial sleeve 5. Rotation of the clutch sleeve 9 causes the drive member 15 to rotate due to their splined engagement. The spline insert 14 rotationally couples the driven member 13 to the housing 1 and therefore the driven member 13 then advances due to the overhauling of its threaded engagement with the drive member 15. The dial sleeve 5 rotation further causes the gauge element 4 to traverse axially back to its zero position whereby the zero dose abutment stops the mechanism.

In the dispensing direction, the end tips of the flexible arms on the drive member 15 are rotated against the shallow sides of the ratchet teeth of the body insert 17. This contact causes inward radial deflection of the flexible arms, enabling overhaul of the ratchet. In this situation, the mid tip does not contact the ratchet surfaces due to its radial offset in comparison to the end tip, ensuring the arm is deflected from the end tip. The angle of this ratchet surface and the formulation of the flexible arms are designed to minimise the torque for overhaul, in order to reduce torque losses from the mechanism. This flexible arm mechanism also provides feedback during dose dispense. As the flexible arms are overhauled by the ratchet features and subsequently released they produce an audible click with each dose increment delivered.

Delivery of a dose continues via the mechanical interactions described above while the user continues to depress the button 12. If the user releases the button 12, the clutch spring 16 returns the clutch sleeve 9 to its at rest position together with the button 12, engaging the splines between the button 12 and dial sleeve 5 and therefore re-engaging the dialing clutch, preventing further rotation and stopping dose delivery. During delivery of a dose, the clutch sleeve 9 and the dial sleeve 5 rotate together, so that no relative motion in the last dose nut 10 occurs. The last dose nut 10 therefore travels axially relative to the clutch sleeve 9 during dialing only.

Once the delivery of a dose is stopped, by the dial sleeve 5 returning to the zero dose abutment, the user may release the button 12, which will push the clutch sleeve 9 and the button 12 back proximally under the action of the clutch spring 16. This will de-couple the first clutch interface 12a between the button 12 and the housing 1, re-engage second clutch interface, e.g., the spline teeth 12b between the button 12 and the dial sleeve 5, and therefore re-engaging the dialing clutch and de-couple the fourth clutch interface between the dial sleeve 5 and the clutch sleeve 9. The mechanism is now returned to the at rest condition.

It is possible to angle the spline teeth on either the button 12 or dial sleeve 5 so that when the button 12 is released the re-engagement of the spline teeth fractionally backwinds the dial sleeve 5 thereby removing the engagement of the dial sleeve to the zero dose stop abutment on the gauge component 4. This compensates for the effect of clearances in the mechanism (for example due to tolerances) which could otherwise lead to slight advancement of the driven member 13 and medicament dispense when the device is dialed for the subsequent dose due to the dial sleeve 5 zero dose stop no longer restraining the mechanism and instead the restraint returning to the splines between the button 12 and the dial sleeve 5.

Frictional losses between the drive member 15 and the housing 1 are reduced since the clutch spring 16 diverts some of the axial reaction force applied by the driven member 13 to the drive member 15 during dispense through the clutch sleeve 9 and to the button 12. The interface between the central spigot 12d of the button 12 and a spigot contact face 9b of the clutch sleeve 9 has low frictional loss due to the small diameter of the contact spigot (FIG. 12). Thus, compared with reacting the axial force via the outer diameter of the drive member 15, the frictional loss is significantly reduced.

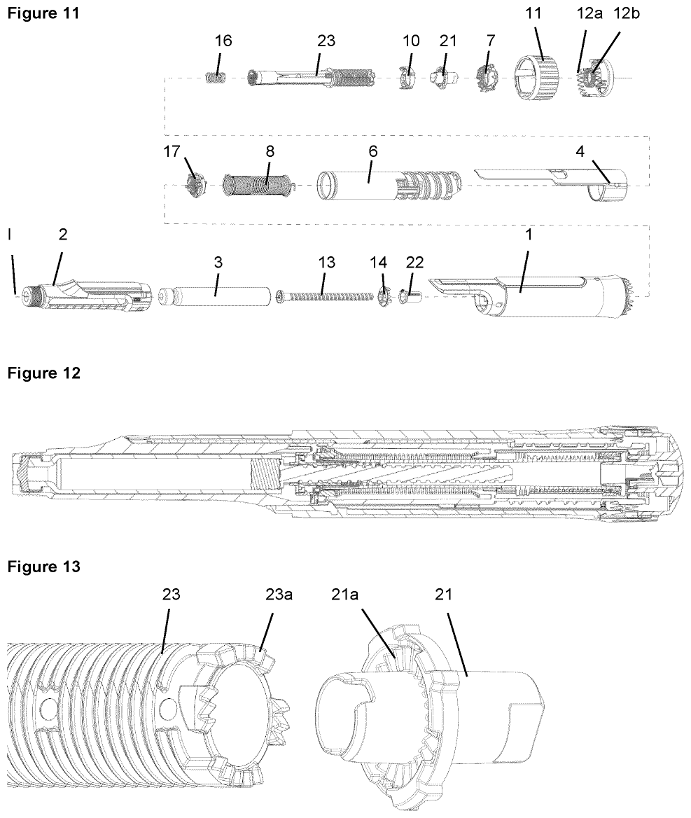

A second embodiment is shown in FIGS. 11 to 14. The general working principle as well as the design and function of the component parts is similar or identical with the above described first embodiment.

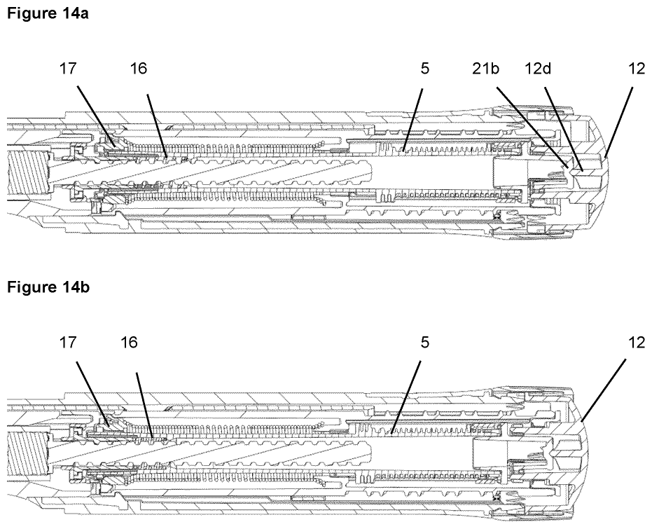

Again, the driven member 13 no longer rotates with respect to the housing 1 (as in some prior art devices) allowing an integrated bearing (pressure foot) in the driven member and therefore improving buckling strength. The spline insert 14 is fixed to the housing 1 and provides rotational constraint to the driven member 13. Compared with the first embodiment, the drive member 22 has rather the form of a collar or ring instead of the sleeve-like configuration of the drive member 15 of the first embodiment. However, the drive member 22 which is rotated during dispense is still threaded to the driven member 13 and axially fixed to the housing 1. Further, it is rotationally splined to the clutch sleeve 23 which is longer in the second embodiment compared with the first embodiment. The clutch spring 16 is situated inside the clutch sleeve 23 and acts axially between the drive member 22 and the clutch sleeve 23 as described above. Frictional losses between the drive member 22 and the housing 1 are reduced since the clutch spring 16 diverts some of the axial reaction force applied by the driven member 13 to the drive member 22 during dispense through a separate, e.g., ring-shaped, clutch plate 21 and to the button 12. This interface between the central spigot 12d of the button 12 and a spigot contact face 21b of the clutch plate 21 has low frictional loss due to the small diameter of the contact spigot (FIG. 14a). Thus, compared with reacting the axial force via the outer diameter of the drive member 22, the frictional loss is significantly reduced.

Further, the second embodiment differs from the first embodiment in that the third clutch interface is provided directly between the clutch sleeve 23 (instead of the drive member) and the body insert 17. For this purpose, the clutch sleeve is provided with teeth 23b at its distal end and body insert 17 is provided with a toothed ring 20 engaging with the teeth 23b depending on the axial position of the clutch sleeve 23, e.g., if the button 12 is depressed in the dispensing mode (de-coupling the third clutch interface 23b, 20) or is released (coupling the third clutch interface 23b, 20).

The clutch plate 21 is splined to the dial sleeve 5. It is also coupled to the clutch sleeve 23 via a detent interface. This detent provides a detented position between the dial sleeve 5 and the clutch sleeve 23 corresponding to each dose unit, and engages different ramped tooth angles during clockwise and anti-clockwise relative rotation. The axial position of the clutch sleeve 23, the clutch plate 21 and the button 12 is defined by the action of the clutch spring 16 which applies a force to the clutch sleeve 23 in the proximal direction.

During dose setting, the clutch sleeve 23 is prevented from rotating as the dial sleeve 5 rotates due to the engagement of its splined teeth with the body insert 17. Relative rotation ca n therefore occur between the clutch plate 21 driven by the dial sleeve 5 and the clutch sleeve 23 via the ratchet interface. This ratchet interface may be provided by ratchet teeth 23a located on the proximally facing end of the clutch sleeve 23 and corresponding ratchet teeth 21a located on a distally facing side of a flange of the clutch plate 21 (FIG. 13).

As depicted in FIGS. 14a, 14b, the coupling and de-coupling of the first clutch interface between the button 12 and the housing 1, of the second clutch interface between the button 12 and the dial sleeve 5, of the third clutch interface between the clutch sleeve 23 and the housing 1 (or the body insert 17 with its toothed ring 20) and of the fourth clutch interface between the clutch sleeve 9 and the dial sleeve 5 occurs in a similar way as described above for the first embodiment upon actuation of the button 12 (switching from the dose setting mode to the dose dispensing mode) or when releasing the button 12 under the action of clutch spring 16 switching the mechanism back from the dose dispensing mode to the dose setting mode.

Reference numerals:

| | |
|---|---|
| 1 | stationary housing (body) |
| 1a | teeth |
| 1b | pocket |
| 2 | cartridge holder |
| 3 | cartridge |
| 4 | gauge component |
| 5 | dial sleeve |
| 6 | lower portion of dial sleeve 5 |
| 7 | upper portion of dial sleeve 5 |
| 8 | drive spring (torsion spring) |
| 9 | clutch sleeve |
| 9a | splines |
| 9b | spigot contact face |
| 10 | nut |
| 11 | dial grip |
| 11a | toothed surface |
| 12 | button |
| 12a | splines |
| 12b | teeth |
| 12c | stem |
| 12d | spigot |
| 13 | driven member |
| 14 | spline insert |
| 14a | teeth |
| 15 | drive member |
| 15a | arms |
| 15b | splines |
| 16 | clutch spring |
| 17 | body insert |
| 18 | one-way ratchet |
| 19 | ratchet element (detent pressing) |
| 19a | hook |
| 19b | nib |
| 19c | bend |
| 20 | toothed ring |
| 21 | separate ring (clutch plate) |
| 21a | ratchet teeth |
| 21b | spigot contact face |
| 22 | drive member |
| 23 | clutch sleeve |
| 23a | ratchet teeth |
| 23b | teeth |
| I | axis |

1-15. (canceled)

16. A dose setting and drive mechanism for a drug delivery device, the dose setting and drive mechanism comprising:
a stationary housing;
a dial sleeve axially constrained to the stationary housing and rotatable relative to the stationary housing; and
a button that is configured to switch between a dose setting mode and a dose dispensing mode, wherein the button comprises:
a first clutch interface that is configured to rotationally couple and de-couple the button and the stationary housing; and
a second clutch interface that is configured to rotationally couple and de-couple the button and the dial sleeve;
a driven member that is axially movable relative to the stationary housing;
a drive member axially constrained to the stationary housing and coupled to the driven member in a manner such that rotation of the drive member in a dose dispensing direction is translated into axial displacement of the driven member; and
a clutch sleeve rotationally constrained and axially movable relative to the drive member, wherein:
the drive member or the clutch sleeve comprises a third clutch interface that is configured to prevent rotation of the drive member and the clutch sleeve relative to the stationary housing in a direction opposite to the dose dispensing direction,
the clutch sleeve comprises a fourth clutch interface that is configured to rotationally couple and de-couple the clutch sleeve and the dial sleeve,
in the dose setting mode, the button is rotationally de-coupled from the stationary housing by the first clutch interface, the button is rotationally coupled to the dial sleeve by the second clutch interface, and the clutch sleeve is de-coupled from the dial sleeve by the fourth clutch interface, and
in the dose dispensing mode, the button is rotationally coupled to the stationary housing by the first clutch interface, the button is rotationally de-coupled from the dial sleeve by the second clutch interface, and the clutch sleeve is coupled to the dial sleeve by the fourth clutch interface.

17. The dose setting and drive mechanism of claim 16, wherein the drive member and the clutch sleeve comprise axially extending grooves and splines that are configured to permanently rotationally constrain the clutch sleeve to the drive member and permit relative axial movement therebetween.

18. The dose setting and drive mechanism of claim 16, further comprising a clutch spring interposed between the drive member and the clutch sleeve in a manner such that the clutch sleeve is biased proximally with respect to the drive member.

19. The dose setting and drive mechanism of claim 16, wherein the driven member is rotationally constrained to the stationary housing and coupled to the drive member by a threaded interface.

20. The dose setting and drive mechanism of claim 16, wherein distal movement of the button is transmitted to the clutch sleeve and proximal movement of the clutch sleeve is transmitted to the button.

21. The dose setting and drive mechanism of claim 16, further comprising a drive spring interposed between the stationary housing and the dial sleeve.

22. The dose setting and drive mechanism of claim 16, further comprising a dial grip that is configured to set a dose, wherein the button is rotationally constrained and axially displaceable relative to the dial grip and wherein the dial grip is axially constrained to the stationary housing.

23. The dose setting and drive mechanism of claim 22, further comprising a fifth clutch interface comprising an overhaulable ratchet element that is configured to rotationally couple the stationary housing and the dial grip, wherein, in the dose setting mode, a driving torque or a driving force of the drive spring is reacted via the fifth clutch interface.

24. The dose setting and drive mechanism of claim 23, wherein the ratchet element is a detent rotationally constrained to the stationary housing and engaging a series of discrete detent positions defined in the dial grip.

25. The dose setting and drive mechanism of claim 16, wherein the fourth clutch interface is configured to rotationally couple and de-couple the clutch sleeve, and the dial sleeve comprises a ring of teeth or splines formed on the clutch sleeve, and a corresponding ring of teeth formed on the dial sleeve.

26. The dose setting and drive mechanism of claim 16, further comprising an insert with a one-way ratchet rotationally constrained to the stationary housing, wherein the one-way ratchet is the third clutch interface allowing rotation of the drive member relative to the stationary housing in the dose dispensing direction and preventing rotation of the drive member relative to the stationary housing in the direction opposite the dose dispensing direction.

27. The dose setting and drive mechanism of claim 16, wherein the button directly abuts the clutch sleeve.

28. The dose setting and drive mechanism of claim 16, further comprising a toothed ring rotationally constrained to the stationary housing, wherein the toothed ring is part of the third clutch interface rotationally coupling the clutch sleeve to the stationary housing in the dose setting mode and rotationally de-coupling the clutch sleeve from the stationary housing in the dose dispensing mode.

29. The dose setting and drive mechanism of claim 16, wherein the fourth clutch interface is configured to rotationally couple and de-couple the clutch sleeve, and the dial sleeve comprises a separate ring which is rotationally constrained to the dial sleeve and comprises a ring of teeth that are configured to engage a corresponding ring of teeth formed on the clutch sleeve.

30. A disposable drug delivery device for selecting and dispensing a number of user variable doses of a medicament, comprising:
a cartridge comprising the medicament, wherein the cartridge is selectively removable;
a stationary housing, wherein the cartridge is configured to fit within the stationary housing;
a dial sleeve axially constrained to the stationary housing and rotatable relative to the stationary housing; and
a button that is configured to switch between a dose setting mode and a dose dispensing mode, wherein the button comprises:
a first clutch interface that is configured to rotationally couple and de-couple the button and the stationary housing; and
a second clutch interface that is configured to rotationally couple and de-couple the button and the dial sleeve;
a driven member that is axially movable relative to the stationary housing;
a drive member axially constrained to the stationary housing and coupled to the driven member in a manner such that rotation of the drive member in a dose dispensing direction is translated into axial displacement of the driven member; and
a clutch sleeve rotationally constrained and axially movable relative to the drive member, wherein:
the drive member or the clutch sleeve comprises a third clutch interface that is configured to prevent rotation of the drive member and the clutch sleeve relative to the stationary housing in a direction opposite to the dose dispensing direction, and
the clutch sleeve comprises a fourth clutch interface that is configured to rotationally couple and de-couple the clutch sleeve and the dial sleeve.

31. The disposable drug delivery device of claim 30, wherein the cartridge comprises two or more chambers that are configured to respectively store two or more components of the medicament.

32. The disposable drug delivery device of claim 31, wherein the two or more chambers of the cartridge are configured to allow mixing between the two or more components of the medicament prior to dispensing into a subject.

33. The disposable drug delivery device of claim 31, wherein the two or more chambers of the cartridge are configured to allow mixing between the two or more components of the medicament during dispensing into a subject.

34. The disposable drug delivery device of claim 31, further comprising a dial grip that is configured to set a dose of the medicament, wherein the button is rotationally constrained and axially displaceable relative to the dial grip and wherein the dial grip is axially constrained to the stationary housing.

35. The disposable drug delivery device of claim 31, further comprising an insert with a one-way ratchet rotationally constrained to the stationary housing, wherein the one-way ratchet is and a third clutch interface allowing rotation of the drive member relative to the stationary housing in the dose dispensing direction and preventing rotation of the drive member relative to the stationary housing in the direction opposite the dose dispensing direction.
